(19) United States
(12) Patent Application Publication
(10) Pub. No.: US 2011/0245510 A1
(43) Pub. Date: Oct. 6, 2011

(54) PROCESS FOR REMOVING BY-PRODUCTS FROM N-VINYLAMIDES

(75) Inventors: Wolfgang Staffel, Waldsee (DE); Regina Vogelsang, Ludwigshafen (DE); Roland Kessinger, Weinheim (DE); Lembit Tuttelberg, Mannheim (DE); Bernd Heida, Ellerstadt (DE)

(73) Assignee: BASF SE, LUDWIGSHAFEN (DE)

(21) Appl. No.: 13/129,544

(22) PCT Filed: Nov. 12, 2009
(86) PCT No.: PCT/EP09/65038

§ 371 Date: May 16, 2011

(30) Foreign Application Priority Data
Nov. 28, 2008 (EP) 08170175.7

(51) Int. Cl. C07D 207/267; C07C 231/24

(52) U.S. Cl. 548/555; 564/216

(57) ABSTRACT

A process for removing by-products from N-vinylamide-rich product mixtures (crude N-vinylamide), which comprises performing an extraction of the crude N-vinylamide with an organic solvent as the extractant.

The invention relates to a process for removing by-products from N-vinylamide-rich product mixtures (crude N-vinylamide), which comprises performing an extraction of the crude N-vinylamide with an organic solvent as the extractant.

Preparation processes for N-vinylamides, for example including N-vinylpyrrolidone, are described, for example, in DE-A 102 55 437, DE-A 195 09 362 and DE-A 198 39 565. As a result of the preparation processes, the product mixture obtained comprises a series of different by-products, for example starting materials, oligomers or polymers which have already formed from the vinyl compounds and other by-products, which can form especially in the course of performance of the chemical conversion in the reactor. The latter also include fluorescent by-products which are removed only incompletely, if at all, in the course of a customary distillative workup of the product mixture.

These fluorescent by-products reduce the product quality. What is desired is therefore a simple process for very substantially removing these fluorescent by-products from the product mixture.

Accordingly, the process defined at the outset has been found.

In the process according to the invention, by-products are removed from N-vinylamide-rich product mixtures (crude N-vinylamide) by extraction.

The crude N-vinylamide

The N-vinylamide may comprise cyclic vinylamides (vinyllactams) or noncyclic N-vinylamides, for example those of the formula

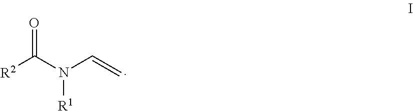

In formula I, R1 and R2 are each independently a hydrogen atom or a C1 to C10 alkyl group, preferably a C1 to C4 alkyl group.

Noncyclic N-vinylamides of the formula I include especially N-vinylformamide (R1 and R2=H) and N-vinyl-N-methylacetamide (VIMA, R1 and R2=methyl).

Vinyllactams include especially N-vinylpiperidone, N-vinylcaprolactam, N-vinyl-pyrrolidone or derivatives thereof. The derivatives are especially vinyllactams in which one or more, preferably one or two, carbon atoms of the ring system are substituted by C1 to C10 alkyl groups; examples include especially N-vinylmethylpyrrolidone, N-vinyldimethylpyrrolidone or N-vinylethylpyrrolidone.

Particularly preferred N-vinylamides are vinyllactams, especially N-vinylcaprolactam or N-vinylpyrrolidone.

A very particularly preferred N-vinylamide is N-vinylpyrrolidone.

According to the invention, a crude N-vinylamide is extracted.

Crude N-vinylamide is understood to mean an N-vinylamide-rich product mixture. This may be the product mixture which forms as the product of the chemical conversion in the reactor, is subsequently withdrawn as reactor discharge and is fed directly to the extraction. In the case of N-vinylpyrrolidone, this is preferably the product mixture which is obtained in the reaction of 2-pyrrolidone (gamma-butyrolactam) with acetylene.

However, the crude N-vinylamide may also be from other preparation processes, as described, for example, in WO 2006/109869 on page 5. Crude N-vinylamide is obtainable, for example, by reaction of butyrolactone with ethanolamine, substitution of the hydroxy group with a chloride using thionyl chloride and subsequent dehydration, or by reaction of N-(2-hydroxyethyl)-2-pyrrolidone with acetic anhydride and elimination of acetic acid, or by gas phase dehydration of N-hydroxyethyl-2-pyrrolidone.

The crude N-vinylamide used for the extraction may also be a product mixture which has already been worked up further after the conversion in the reactor, or starting materials or by-products.

More particularly, the crude N-vinylamide, for example the crude N-vinylpyrrolidone, is a mixture which was obtained by reacting starting materials, for example 2-pyrrolidone, with acetylene and optionally a subsequent workup of the product mixture obtained.

Generally, the mixture used as the crude N-vinylamide for the extraction consists of the N-vinylamide to an extent of at least 50% by weight, more preferably to an extent of at least 70% by weight and especially to an extent of at least 80% by weight.

In the case of the N-vinylpyrrolidone, particular preference is given to using a crude N-vinylpyrrolidone which was first obtained by reacting acetylene with 2-pyrrolidone and from which high boilers (for example oligomers of N-vinylpyrrolidone) were then removed by a first distillation (crude distillation). The mixture distilled off comprises N-vinylpyrrolidone, generally in amounts greater than 70% by weight, more preferably greater than 80% by weight, and residual amounts of 2-pyrrolidone (generally 0 to 30% by weight, more preferably 1 to 25% by weight) and by-products with a boiling point above 2-pyrrolidone, generally in amounts of 0.5 to 20% by weight, more preferably 0.5 to 10% by weight, all above weight data being based on the mixture. In a particularly preferred embodiment, this mixture is used as the crude N-vinylpyrrolidone for the subsequent extraction.

The crude N-vinylamide, for example crude N-vinylpyrrolidone, used for the extraction comprises especially fluorescent by-products. These by-products are possibly aromatic compounds with a boiling point between 150 and 320° C., especially 200 to 260° C. (at standard pressure).

### The Extractant

In the extraction, an organic solvent is used as the extractant.

The by-products for removal should have maximum solubility in the solvent. At the same time, the solvent must have only low miscibility, if any, with the mixture to be extracted under the extraction conditions, such that two phases form. The phase diagram of the binary system composed of mixture to be extracted and extractant must have a miscibility gap under the extraction conditions.

If the crude N-vinylamide does not have a miscibility gap with the extractant, it is possible to use, instead of the crude N-vinylamide, a mixture which has a corresponding miscibility gap. The mixture component should have maximum miscibility with the crude N-vinylamide, have minimum miscibility with the extractant and be readily removable from the crude N-vinylamide after the extraction. A preferred mixture component is water. This may also be water which comprises inorganic or organic salts in dissolved form.

In a preferred embodiment of the invention, the extraction is effected with addition of water. In that case, the extractant consists of water and one or more organic solvents. The extractant may consist, for example, to an extent of 1 to 70% by weight, especially 2 to 50% by weight, of water.

Suitable extractants are, for example, aromatic solvents such as benzene, toluene or xylene (isomer mixture). Aromatic solvents are, however, frequently unwanted. In addition, it is also disadvantageous in the present case that N-vinylpyrrolidone is partly transferred to the organic phase and the N-vinylpyrrolidone is therefore not removed completely.

Suitable organic solvents for the extractant are especially aliphatic or cycloaliphatic solvents. Particular preference is given to aliphatic or cycloaliphatic solvents which are liquid at 21° C. (1 bar) and have a boiling point greater than 80° C. (at 1 bar). They are especially completely nonpolar solvents, i.e. compounds which consist only of carbon and hydrogen (hydrocarbons).

The aforementioned aliphatic or cycloaliphatic solvents can of course also be used in a mixture with aromatic solvents. The content of aromatic solvents in the total amount of all organic solvents used in the extractant is more preferably less than 50% by weight, especially less than 30% by weight, more preferably less than 10% by weight, or less than 5% by weight. In a particularly preferred embodiment, the organic solvents used as extractants do not comprise any aromatic solvents.

Suitable aliphatic or cycloaliphatic solvents include, for example, methylcyclopentane, cyclopentane, hexane, methylcyclohexane, cyclohexane, heptane or octane. Suitable solvents are especially compounds which have a miscibility gap with the N-vinylamide to be extracted. It is also possible to use mixtures of different solvents as extractants; examples include mixtures of cyclohexane and methylcyclohexane in any desired ratios.

Particularly preferred organic solvents include methylcyclohexane, cyclohexane, methylcyclopentane and cyclopentane, and mixtures thereof. Very particular preference is given to mixtures of methylcyclopentane and cyclopentane, or mixtures of methylcyclohexane and cyclohexane.

Very particular preference is given to extractants which comprise or consist of water and the above particularly preferred organic solvents.

In a particularly preferred embodiment, an extractant which comprises or consists of methylcyclohexane and water is used as the extractant for crude N-vinylpyrrolidone.

A mixture of crude N-vinylpyrrolidone and water has a miscibility gap with methylcyclohexane under suitable extraction conditions.

Miscibility gaps in ternary systems can typically be determined by varying the concentration and be represented in triangular diagrams.

### The Performance of the Extraction

Generally, a temperature range from 20 to 100° C. under standard pressure (1 bar) is also suitable for the extraction of the crude N-vinylamides. The extraction can also be performed under elevated pressure or reduced pressure. The extractant can be used, for example, in an amount of 20 to 0.5 parts by weight, especially in an amount of 10 to 1 parts by weight, per 1 part by weight of crude N-vinylpyrrolidone.

In the case of the ternary system composed of crude N-vinylpyrrolidone, water and methylcyclohexane, suitable extraction conditions arise within a temperature range from 20 to 100° C., more preferably 30 to 80° C., at 1 bar and with a proportion by weight of 10 to 50% by weight, more preferably 20 to 40% by weight, of crude N-vinylpyrrolidone, 20 to 80% by weight, more preferably 30 to 70% by weight, of methylcyclohexane and 2 to 50% by weight, more preferably 10 to 30% by weight, of water.

Accordingly, the extraction is performed with the ternary system composed of crude N-vinylpyrrolidone, water and methylcyclohexane, preferably observing the above extraction conditions.

The extraction can be performed batchwise or continuously.

In the case of batchwise extraction, in the simple case, a stirred vessel for mixing the two phases (extractant and phase to be extracted) and a separator in which the two phases separate according to their density into an upper phase and lower phase (mixer-settler) are used. Thereafter, the solvent can be purified, for example by distillation, and be recycled again into the mixer.

The extraction is preferably performed continuously. More particularly, the entire preparation process, comprising the conversion of the starting materials in the reactor and subsequent workup, is performed continuously.

Equally suitable for continuous performance of the extraction are mixer-settlers as described above, or cascades of mixer-settlers; in a preferred embodiment, in the case of continuous performance, columns are used, for example customary extraction columns, such as spray tray columns, sieve tray columns, extraction columns with random packing, pulsed columns or rotary columns, are suitable. The starting components are supplied via what are known as distributors. Distributors are internals in the column which bring about maximum distribution of the components over the column cross section. Suitable distributors are preferably installed horizontally into the columns.

The continuous extraction can be performed in one or more columns. For the ternary crude N-vinylpyrrolidone/water/methylcyclohexane system, one option is, for example, performance of the extraction in an extraction column and a wash column connected thereto. In the extraction column, the undesired by-products are removed from the water/crude N-vinylpyrrolidone mixture with methylcyclohexane; in the wash column, N-vinylpyrrolidone which has likewise (undesirably) been transferred to the methylcyclohexane phase is recovered again with water. The water/N-vinylpyrrolidone mixture removed in the wash column can be recycled into the extraction column.

In a preferred embodiment, the continuous extraction is performed in a column.

The crude NVP, the extractant and optionally the water can be supplied at any points in the column or columns. Crude NVP, extractant and optionally water can be supplied separately or together; it is possible to supply all three components or two of the three components together anywhere in the columns.

The extractant (methylcyclohexane) is preferably supplied via a distributor in the lower part of the column, and water via a distributor in the upper part of the column, while the crude N-vinylpyrrolidone to be extracted is supplied in the middle part. According to the density of the components, the methylcyclohexane extractant rises to the top of the column and is enriched with the by-products to be extracted from the crude N-vinylpyrrolidone and also with N-vinylpyrrolidone; in the upper part of the column, N-vinylpyrrolidone is scrubbed out of the methylcyclohexane with water, such that a substantially N-vinylpyrrolidone-free mixture of methylcyclohexane and extracted by-products can be drawn off at the top. At the bottom of the column, a mixture of purified crude N-vinylpyrrolidone and water is drawn off.

The temperature profile which is established in the column depends on the mixing enthalpies of the components and the distribution of the components in the column. In the case of the ternary crude N-vinylpyrrolidone/water/methylcyclohexane system, the temperature in the column is preferably 10 and 90° C., especially 20 to 60° C. and more preferably 30 to 50° C.

Methylcyclohexane can then be purified by distillation and reused.

The water/crude N-vinylpyrrolidone mixture can also be worked up further.

The crude N-vinylamide purified by extraction, especially crude N-vinylpyrrolidone, now comprises barely any fluorescent by-products. After removal of the water, the content is less than 0.5 part by weight, especially less than 0.1 part by weight and more preferably less than 0.05 part by weight or less than 0.01 part by weight, based on 100 parts by weight of N-vinylpyrrolidone.

### EXAMPLES

N-vinylpyrrolidone was prepared by reacting acetylene with 2-pyrrolidone. High boilers were removed by distillation from the mixture obtained. The top product obtained is an N-vinylpyrrolidone which still comprises fluorescent impurities. This was used as the crude N-vinylpyrrolidone in the downstream extraction tests.

200 milliliters of crude N-vinylpyrrolidone (crude NVP), 200 milliliters of water and 200 milliliters in each case of one of the solvents listed in table 1 as an extractant were mixed and shaken vigorously. An aqueous phase and an organic phase were obtained. The phases were separated and the fluorescence of the aqueous phase which comprises the N-vinylpyrrolidone (NVP extracted) was determined.

The fluorescence (F) determined in the crude N-vinylpyrrolidone was expressed as a ratio to the fluorescence of the aqueous phase obtained:

F═F crude NVP/F NVP extracted.

The greater the value of F, the better the removal achieved of the fluorescent impurities. The value for F is listed in table 1.

The measurements were carried out with a Perkin Elmer LS50-B luminescence spectrophotometer. The substance to be examined was introduced into a UV cuvette (d=1.0 cm), and the wavelength at which fluorescence is at a maximum was determined at three different excitation wavelengths. The intensity of this maximum fluorescence was expressed as a ratio to the intensity of the maximum fluorescence of the crude NVP.

Temperature: 20° C.

Excitation wavelengths λex=320 nm, 340 nm and 360 nm

Gap width for excitation wavelength/emission wavelength: 5 nm/5 nm

TABLE 1
Fluorescence data

| | | Intensity of | | Intensity of | | Intensity of | | |
|---|---|---|---|---|---|---|---|---|
| | | emission | | emission | | emission | | |
| | | maximum at | | maximum at | | maximum at | | |
| | Extraction | λex = 320 nm | | λex = 340 nm | | λex = 360 nm | | |
| | solvent | absolute | F | absolute | F | absolute | F | Comment |
| 1 | none | 3470* | 1 | 5208* | 1 | 3558* | 1 | |
| 2 | benzene | 246 | 14.1 | 341 | 15.3 | 168 | 21.2 | Some of the |
| | | | | | | | | NVP remained |
| | | | | | | | | in the organic |
| | | | | | | | | phase |
| 3 | toluene | 211 | 16.4 | 278.2 | 18.7 | 133.6 | 26.6 | Some of the |
| | | | | | | | | NVP remained |
| | | | | | | | | in the organic |
| | | | | | | | | phase |
| 4 | xylene | 269 | 12.9 | 258.8 | 20.1 | 123.5 | 28.8 | Some of the |
| | (isomer | | | | | | | NVP remained |
| | mixture) | | | | | | | in the organic |
| | | | | | | | | phase |
| 5 | hexane | 770 | 4.5 | >1000 | <5 | >1000 | <5 | |
| 6 | heptane | 749.2 | 4.6 | 587 | 8.9 | 622.2 | 5.7 | |
| 7 | octane | 753 | 4.6 | 553 | 9.4 | 566 | 6.3 | |
| 8 | petroleum | 357 | 9.7 | 680 | 7.7 | 419 | 8.5 | |
| | ether | | | | | | | |
| 9 | methylcyclo- | 722 | 4.8 | 392 | 13.3 | 351 | 10.1 | |
| | pentane | | | | | | | |
| 10 | methylcyclo- | 690 | 5.0 | 387 | 13.5 | 360 | 9.9 | |
| | hexane | | | | | | | |
| 11 | cyclohexane | 750 | 4.6 | 410 | 12.7 | 379 | 9.4 | |
| 12 | diethyl ether | 714 | 4.9 | 950 | 5.5 | >1000 | <5 | |
| 13 | tert-butyl | 801 | 4.3 | 916 | 5.7 | 975 | 3.7 | |
| | methyl ether | | | | | | | |

*For the fluorescence measurement, 1 part by weight of crude NVP was diluted with 20 parts by weight of water.

1. A process for removing by-products from a N-vinylamide-rich product mixture, which comprises extracting the crude N-vinylamide with an organic solvent as the extractant.

2. The process according to claim 1, wherein the N-vinylamide comprises cyclic N-vinylamides (vinyllactams) or noncyclic N-vinylamides of the formula 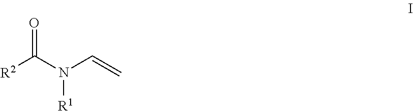
in which R1 and R2 are each independently a hydrogen atom or a C1 to C10 alkyl group.

3. The process claim 1, wherein the noncyclic N-vinylamides of the formula I comprise N-vinylformamide, wherein (R1 and R2=H, or N-vinyl-N-methylacetamide, and VIMA, R1 and R2=methyl.

4. The process according to claim 1, wherein the cyclic N-vinylamide is N-vinylpiperidone, N-vinyl-caprolactam or N-vinylpyrrolidone or derivatives thereof.

5. The process according to claim 1, wherein the N-vinylamide is N-vinylpyrrolidone.

6. The process according to claim 1, wherein the crude N-vinylamide consists of the N-vinylamide to an extent of at least 50% by weight.

7. The process according to claim 1, wherein the crude N-vinylamide was obtained by a process comprising reacting starting materials with acetylene and optionally a subsequent workup.

8. The process according to claim 1, wherein the crude N-vinylamide is crude N-vinylpyrrolidone which was obtained by a process comprising reacting 2-pyrrolidone (gamma-butyrolactam) with acetylene and optionally a subsequent workup.

9. The process according to claim 1, wherein the extraction is preceded by distillation to remove the high boilers from the crude N-vinylamide.

10. The process according to claim 1, wherein the solvent is an aliphatic or cycloaliphatic solvent.

11. The process according to claim 1, wherein the extraction is effective with addition of water.

12. The process according to claim 1, wherein crude N-vinylpyrrolidone is extracted with methylcyclohexane with addition of water.

13. The process according to claim 1, wherein the extraction is performed within a temperature range from 20 to 100° C.

14. The process according to claim 1, wherein the extraction is performed continuously in one or more columns.

15. The process according to claim 1, wherein crude N-vinylpyrrolidone is extracted continuously with methylcyclohexane with addition of water in a column, water being supplied via a distributor in the upper part of the column, methylcyclohexane via a distributor in the lower part of the column and the crude N-vinylpyrrolidone in the middle part of the column.

16. The process according to claim 15, wherein a mixture of methylcyclohexane and removed by-products is withdrawn at the top of the column, and a mixture of purified crude N-vinylpyrrolidone and water at the bottom of the column.

17. The process according to claim 1, wherein the extraction removes fluorescent by-products from the crude N-vinylamide down to a content of less than 0.05 part by weight, based on 100 parts by weight of N-vinylpyrrolidone.

18. The process according to claim 1, wherein the cyclic N-vinylamide is at least one selected from the group consisting of N-vinylmethylpyrrolidone, N-vinyldimethylpyrrolidone and N-vinylethylpyrrolidone.
